(19) United States
(12) Patent Application Publication
(10) Pub. No.: US 2023/0391788 A1
(43) Pub. Date: Dec. 7, 2023

(54) PROCESS FOR PREPARATION OF PURE NALTREXONE DECANOATE, ITS SALTS, COMPOSITION AND METHOD OF USE THEREOF

(71) Applicant: NAVIN SAXENA RESEARCH & TECHNOLOGY PVT. LTD., Mumbai (IN)

(72) Inventors: Kunal SAXENA, Mumbai (IN); Aarkarsh SAXENA, Mumbai (IN)

(21) Appl. No.: 18/250,853

(22) PCT Filed: Oct. 27, 2021
(86) PCT No.: PCT/IN2021/051027

§ 371 Date: Apr. 27, 2023

(30) Foreign Application Priority Data
Oct. 27, 2020 (IN) 202021046814

(51) Int. Cl. C07D 489/09; A61K 47/44; A61K 47/10; A61K 9/00; A61K 47/34; A61K 31/485

(52) U.S. Cl. CPC C07D 489/09; A61K 47/44; A61K 47/10; A61K 9/0019; A61K 47/34; A61K 31/485

(57) ABSTRACT

Disclosed herein is an improved process for preparation of naltrexone decanoate under a monophasic medium using a single solvent by esterifying naltrexone with a decanoyl chloride in the presence of an organic base, wherein the solvent is preferably cyclo-pentyl-methyl-ether (CPME) which is non-toxic in nature. The invention further discloses the elimination of an impurity bis-decanoyl naltrexone obtained during the preparation of naltrexone decanoate by preparing acid addition salts of naltrexone decanoate and reconverting into naltrexone decanoate from its acid addition salts by neutralizing using a base in the presence of suitable solvent to obtain naltrexone decanoate with a purity of more than 99% The invention also discloses pharmaceutical composition of naltrexone decanoate with at least one pharmaceutically acceptable excipient and method of use of naltrexone decanoate for the treatment of opioid dependence, alcohol dependence for a period of 7 days to 90 days in a patient in need thereof.

### FIELD OF INVENTION

This invention relates generally to the synthesis of naltrexone decanoate, synthesis of naltrexone decanoate organic acid addition salts thereof and the process for reconverting naltrexone decanoate from its organic acid addition salts for obtaining highly pure naltrexone decanoate. The present invention also provides a solution for the treatment of alcohol and opioid addiction by using sustained release composition of naltrexone decanoate, which would help to improve patient compliance.

### BACKGROUND OF INVENTION

Naltrexone hydrochloride is an opioid antagonist. The compound and methods for the synthesis of Naltrexone are described in U.S. Pat. No. 3,332,950. Naltrexone hydrochloride (CAS: 16676-29-2) has the molecular formula C₂₀H₂₃O₄·HCl. Naltrexone hydrochloride is chemically known as morphinan-6-one, (5α)-17-(cyclopropylmethyl)-4,5-epoxy-3,14-dihydroxy-, hydrochloride, having the following structural formula:

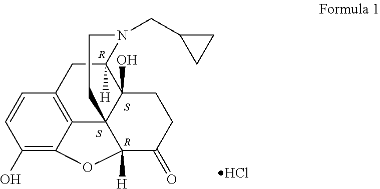

Naltrexone is an opioid receptor antagonist used primarily in the management of alcohol dependence and opioid dependence. The main use of naltrexone is for the treatment of alcohol dependence. It is marketed in generic form as its hydrochloride salt, viz., naltrexone hydrochloride, and marketed under the trade names Revia® and Depade® in the form of 50 mg film coated tablets. In some countries including United States, a once-monthly extended-release injectable formulation is marketed under the trade name Vivitrol®.

Although the usage of naltrexone for the treatment of alcohol dependence and opioid dependence provides a great benefit to the society, however, the problem associated with this drug is that the drug has very short period of action. Thus, for example, well absorbed orally (approximately 96% of an oral dose is absorbed from the gastrointestinal tract), naltrexone is subject to significant first pass metabolism with oral bioavailability estimates ranging from 5% to 40%. The activity ofnaltrexone is believed to be as a result of both naltrexone and its 6-β-naltrexol metabolite. Two other minor metabolites are 2-hydroxy-3-methoxy-6-β-naltrexol and 2-hydroxy-3methyl-naltrexone. Peak plasma levels of both naltrexone and 6-β-naltrexone occur within one hour after oral dosing; mean elimination half-life values for naltrexone and 6-β-naltrexol are four and thirteen hours respectively. Even for long acting naltrexone injections, clinicians indicate that patients discontinue treatment too early. Therefore, a need exists for ultra-long acting opioid antagonists in the treatment of substance abuse disorder.

One of the solutions to overcome the problem of short period of action of naltrexone is to use esters or prodrugs which provide a long, sustained and controlled release of naltrexone opioid receptor antagonist upon administration into the body.

In search for a non-oral delivery route for naltrexone, Stinchcomb and co-workers assessed the human skin permeation of straight-chain alkyl ester and branched-chain alkyl ester and carbonate prodrugs of naltrexone alongside the parent drug from mineral oil in saturated conditions. All the straight-chain ester prodrugs enhanced the skin permeation of naltrexone. In the case of the straight-chain esters, the N-ethyl and N-propyl carbamate prodrugs were the best penetrants, enhancing the permeation of naltrexone upto 3-fold. (Prodrug Strategies for Enhancing the Percutaneous Absorption of Drugs; David D. N'Da; Molecules 2014, 19, 20780-20807).
Naltrexone decanoate is a straight chain ester or prodrug of Naltrexone, which is represented by the IUPAC name: 17-(cyclopropylmethyl)-4, 5-epoxy-14-hydroxy-3-[(1-oxodecyl) oxy]-(5α)-morphinan-6-one, and has the structure:

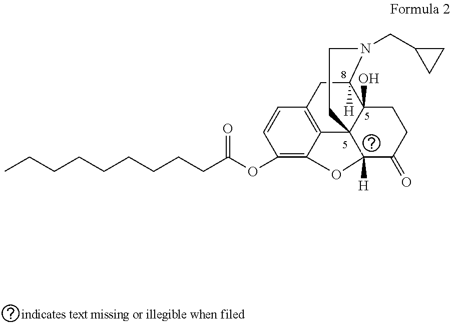 [SMILES: CCCCCCCCCC(=O)Oc1ccc2c3c1O[C@H]1C(=O)CC[C@@]4(O)[C@@H](C2)N(CC2CC2)CC[C@]314]

There are various methods reported for preparing N-substituted-14-hydroxymorphinane esters that are used as narcotic analgesics and opiate receptor antagonists with prolonged effect.

USSR Patent SU1508960 discloses a method for synthesizing benzoate ester derivatives of naloxone or naltrexone. According to which 3-hydroxymorphinane and acid anhydride is dissolved in a dipolar aprotic solvent, for example, N, N-dimethylformamide, dimethyl sulfoxide, N-methylpyrrolidone, N, N dimethyl acetamide in presence of a base for example, 4-dimethylaminopyridine or 4-pyrrolidine pyridine and the reaction mixture is kept heating at 55-60° C. under nitrogen atmosphere. The yield of the target product after double recrystallization is 65-70%. The disclosed process for preparation of naloxone and naltrexone esters including naltrexone decanoate are not commercially viable due to formation of impurity.

Another patent RU 2215742 discloses the synthesis of esters of naltrexone and naloxone using a biphasic medium by reacting naltrexone and naloxone with an acylating agent selected from the group consisting of carboxylic acid chlorides with 3-18 carbon atoms, in an organic solvent, in the presence of an acceptor of hydrogen halide such as an alkali metal carbonate or bicarbonate, characterized in that a 10-35% aqueous solution of N-substituted-14-hydroxymorphinan hydrochloride is reacted and the reaction is carried out in a water-immiscible chlorine-containing aliphatic hydrocarbon medium such as methylene chloride, chloroform, dichloroethane and trichloroethylene in the presence of an aqueous solution of a hydrogen halide acceptor.

US2020048271 discloses the purification of naltrexone or its salts from a reaction mixture wherein cyclopentyl methyl ether is used for the crystallization or precipitation of naltrexone or its salts for extracting naltrexone from the reaction mixture with a purity of 99.5%. The naltrexone base has good solubility in cyclopentyl methyl ether which is used for the isolation of the product from the reaction mixture and the naltrexone base thus obtained is used for the preparation of appropriate salts which are used in dosage forms.

For industrial application purposes, the method disclosed in RU 2215742 has disadvantages of high level of impurities occurred during the process and the removal of such impurities is a tedious process while producing esters of naltrexone using a biphasic medium. This reaction process also uses the solvents such as dichloroethane, chloroform and trichloroethylene which are toxic in nature. CN1050130C discloses Naltrexone acid ester and synthesis thereof; however, purification of these esters is not reported. Moreover, the process reported in this patent employs chloroform for the extraction of the product, which is a hazardous solvent.

Therefore there is a need within the field to improve the method of producing highly pure naltrexone ester or prodrug such as naltrexone decanoate. In particular, there is a need for a method that is readily applicable on a large industrial scale and which avoids the use of toxic solvents in the reaction process, and permits easy isolation of naltrexone in a pure form suitable for its transformation to the final pharmaceutical salt form.

### SUMMARY OF THE INVENTION

In line with the above need, the present invention provides an improved process for the synthesis of ester or prodrug of naltrexone having a moiety of saturated or unsaturated carboxylic acids of 2-18 carbon atoms, under a monophasic medium using a single solvent by esterifying naltrexone with an acylating agent in the presence of an organic base, which serves as hydrogen acceptor, under suitable conditions such as trimethylamine or Hunig's base, wherein the solvent is preferably non-toxic in nature.

Accordingly, the process for the preparation of ester or prodrug of naltrexone having a moiety of saturated or unsaturated carboxylic acids of 2-18 carbon atoms, comprising the steps of
a. reacting naltrexone with an acylating agent selected from the group consisting of chlorides of saturated or unsaturated carboxylic acids having 2-18 carbon atoms in presence of an organic base and an organic solvent to obtain ester or prodrug of naltrexone having a moiety of saturated or unsaturated carboxylic acids of 2-18 carbon atoms;
b. converting the naltrexone ester or prodrug into its acid addition salt by treating with an acid in an organic solvent to obtain crude ester or prodrug of naltrexone acid addition salt; and
c. neutralizing the acid addition salt of naltrexone ester or prodrug by treating with a base to recover naltrexone ester or prodrug.

In an embodiment, the acylating agent is selected from the group consisting of decanoyl chloride, palmitoyl chloride, stearoyl chloride, myristoyl chloride and sebacoyl chloride.

In one embodiment, there is no particular restriction on the nature of the solvent to be employed for the formation of the compound ester or prodrug of naltrexone, provided that it has no adverse/toxic effect on the reaction or the reagents involved. Examples of suitable solvents include, but are not limited to, ether solvents, and non-polar solvents for example, diethyl ether, tetrahydrofuran, 2-methyl tetrahydrofuran, methyl tert-butyl ether, dimethoxyethane, cyclopentyl methyl ether and diisopropylether. Examples of suitable solvents may also include, but are not limited to, polar aprotic solvents, for example, dichloromethane, acetonitrile, dimethyl fumarate, dimethyl sulfoxide, N-methylpyrrolidinone, dimethylacetamide, diethanolamine, sulfolane, acetone, methyl ethyl ketone and methyl isobutyl ketone. Preferably, the solvent is cyclo pentyl methyl ether (CPME).

The preferred solvent, cyclo pentyl methyl ether is non-hazardous and eco-friendly solvent which results in high yield and high purity of the product. In the present invention, the reaction of naltrexone with acylating agent is preferably carried out by using cyclo pentyl methyl ether instead of the chlorinated solvents used in the prior art processes.

The ester or prodrug of naltrexone is selected from the group consisting of naltrexone isobutyrate, naltrexone pivaloate, naltrexone propionate, naltrexone stearate, naltrexone myristate, naltrexone decanoate and naltrexone sebacate. After the reaction, water was added to reaction mixture, then aqueous layer and organic layer were separated wherein the aqueous layer was separated with dichloromethane and the organic layer was washed with water and brine solution, dried over anhydrous magnesium sulphate or anhydrous sodium sulphate, and concentrated typically under reduced pressure, to provide ester or prodrug of naltrexone.

The ester or prodrug of naltrexone according to the invention is selected from the group consisting of naltrexone isobutyrate, naltrexone pivaloate, naltrexone propionate, naltrexone stearate, naltrexone myristoate, naltrexone decanoate and naltrexone sebacate.

In one preferred embodiment, the ester or prodrug of naltrexone is naltrexone decanoate.

In an aspect, the step of neutralizing ester or prodrug of naltrexone comprises treating the organic acid addition salt of naltrexone ester or prodrug with an inorganic base in non-polar organic solvent at a temperature in the range of 10° C. to 35° C. for a period of 15 minutes to 1 hour to isolate a highly pure ester or prodrug of naltrexone.

The naltrexone decanoate obtained by the process of the present invention is in the form of a viscous oil, wherein the yield of naltrexone decanoate obtained is more than about 90% with an HPLC purity of more than about 99%. In this process, the main impurity coming from the alkylation of the hydroxyl group of naltrexone is bis-decanoyl naltrexone. The level of the impurity bis-decanoyl naltrexone in the isolated naltrexone decanoate is measured by HPLC.

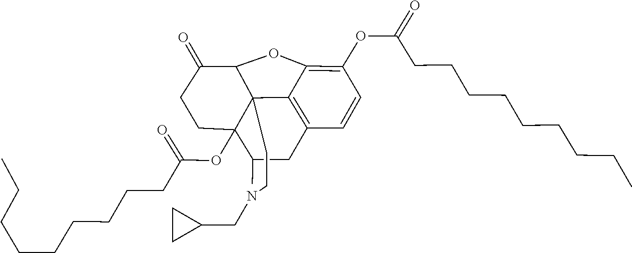 [SMILES: CCCCCCCCCC(=O)Oc1ccc2c3c1OC1C(=O)CCC(OC(=O)CCCCCCCCC)(C(C)C2)C31CCNCC1CC1]

In another embodiment, the invention provides a purification process for elimination of bis-decanoyl naltrexone impurity obtained which process comprising the steps of:
1) reacting naltrexone with decanoyl chloride to obtain naltrexone decanoate;
2) treating decanoyl chloride to obtain naltrexone decanoate with an acid to obtain acid addition salts of naltrexone decanoate; and
3) reconverting to naltrexone decanoate from its acid addition salts by neutralizing the organic acid addition salts using a base in the presence of suitable solvent for obtaining substantially pure naltrexone decanoate which is free of bis-decanoyl naltrexone and with an HPLC purity of more than 99.7% and preferably with an HPLC purity of about 99.9%.

The naltrexone decanoate thus obtained is stable over a period upto 60 months at a storage condition of less than 25° C.

In another embodiment, the present invention provides a process for the purification of naltrexone decanoate. This process comprises the steps of:
a) forming a naltrexone decanoate salt from naltrexone decanoate by addition of an acid to crude naltrexone decanoate,
b) optionally, recovering naltrexone decanoate organic acid addition salts,
c) neutralising naltrexone decanoate acid addition salt by treating with a base until a pH between about 8-10 is reached,
d) extracting naltrexone decanoate with organic solvent,
e) collecting naltrexone decanoate, and
f) optionally washing, re-precipitating or re-crystallising the purified naltrexone decanoate.

In another embodiment, the organic acid addition salts of naltrexone decanoate can be prepared through the reaction with a suitable organic acid in the presence of suitable solvents.

In another embodiment, suitable organic acids for preparing acid addition salts may include, but not limited to acetic acid, propionic acid, glycolic acid, pyruvic acid, oxalic acid, decanoic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluene sulfonic acid and salicylic acid.

In another embodiment, suitable solvent includes polar protic solvent, polar aprotic solvent or non-polar solvent or a mixture of solvents thereof.

In another embodiment, the polar protic solvent may include but not limited to methanol, ethanol, n-butanol, isopropanol, n-propanol, ethyl acetate, tetrahydro furan, acetonitrile, acetone, dimethyl formamide and dichloromethane or mixture(s) thereof.

In another embodiment, the non-polar solvent may include but not limited to dichloromethane, diethyl ether, methyl tertiary-butyl ether, cyclo pentyl methyl ether (CPME), cyclopentyl ethyl ether, cyclopentylpropyl ether, 1,4-dioxane, toluene, pentane, cyclopentane, hexane, cyclohexane, hexane, heptane or mixture(s) thereof.

In another embodiment, the acid addition salt of naltrexone decanoate thus obtained was reconverted to naltrexone decanoate with substantial purity and yield by treating with a suitable base in a non-polar organic solvent. In other embodiment, the base may include an organic base or inorganic base. Preferably an inorganic base is used.

After the reaction, the organic layer was separated and washed with water. The resulting organic layer was dried over sodium sulphate and concentrated under reduced pressure to obtain pure naltrexone decanoate in the form of pale yellow viscous oil. The naltrexone decanoate is obtained in the yield of more than 70% and with an HPLC purity of more than 99%, preferably with an HPLC purity of about 99.9%.

In another embodiment, the inorganic base may include but not limited to hydroxides of alkali metal or alkaline earth metals such as sodium hydroxide, potassium hydroxide, lithium hydroxide, calcium hydroxide, barium hydroxide; bicarbonates of alkali metals such as sodium bicarbonate, potassium bicarbonate or mixture(s) thereof.

In another embodiment, the organic base may include but not limited to pyridine, triethylamine, tripropylamine, tributylamine, N,N-diisopropylethylamine, N-methylmorpholine, N,N-dimethylaminopyridine), and mixtures thereof.

In another embodiment, naltrexone decanoate thus obtained by the process can be formulated into a pharmaceutical composition.

In another embodiment, the pharmaceutical composition of naltrexone decanoate and its pharmaceutically acceptable salts thereof which is used for the treatment of opioid dependence, alcohol dependence, for a period of 7 days to 90 days, wherein the pharmaceutical composition is administered by subcutaneous injection or intramuscular injection.

In another embodiment, the pharmaceutical composition of naltrexone decanoate and its pharmaceutically acceptable salts thereof which is used for the treatment of opioid dependence, alcohol dependence, for a period of 7 days to 90 days, wherein the plasma concentration of Naltrexone remains above 1 ng/mL (the concentration in which Naltrexone imparts de-addiction to alcohol (as per Br J Clin Pharmacol /76:5/632-641, DOI:10.1111/j.1365-2125.2012.04452.x).

A sustained release pharmaceutical composition according to the present invention comprising of naltrexone, its salts and its prodrugs delivering naltrexone over a period of 7 to 90 days in a single dose.

The naltexone or its prodrug is selected from the group consisting of naltrexone acetate, naltrexone butyrate, naltrexone velarate, naltrexone hexanoate, naltrexone heptanoate, naltrexone octanoate, naltrexone nonaoate, naltrexone undecanoate, naltrexone dodecanoate, naltrexone tridecanoate, naltrexone myristate, naltrexone pentadecanoate, naltrexone palmitate, naltrexone heptadecanoate, naltrexone isobutyrate, naltrexone pivaloate, naltrexone propionate, naltrexone stearate, naltrexone decanoate and naltrexone sebacate, and preferably naltrexone decanoate having a concentration in the range of 50 to 1500 mg/ml.

In another embodiment, the present invention provides sustained release pharmaceutical composition of naltrexone decanoate and its pharmaceutically acceptable salt thereof wherein the composition comprises naltrexone decanoate and at least one pharmaceutical excipient selected from;
a) A vehicle in a concentration of about 30.0% w/w to 90.0% w/w of the total composition,
b) A stabilizer in a concentration of about 0.01% w/w to 30.0% w/w of the total composition,
c) A rate limiting polymer in a concentration of about 0.2% w/w to 50% w/w of the total composition.

The vehicle is selected from the group of following or a mixture thereof:
a) Vegetable oils such as cottonseed oil, sesame oil, castor oil, silicon oil, coconut oil, sunflower oil and a mixture thereof
b) Water miscible organic solvent selected from the group consisting of N-methylpyrrolidone, polyethylene glycol, ethanol, a mixture thereof,
c) Water immiscible organic solvent which is selected from benzyl alcohol, benzyl benzoate, and a mixture thereof;

In another embodiment, the stabilizer is selected from the group consisting of butylated hydroxytoluene (BHT), butylated hydroxyanisole (BHA), tocopherol, ascorbic acid, sodium bisulfite and sodium metabisulfite, and a mixture thereof, wherein the stabilizer is present in a concentration of about 0.01% w/w to 30.0% w/w of total composition by weight.

In another embodiment, the rate limiting polymer is selected from the group consisting of poly ortho esters such as poly(lactic-co-glycolic acid) of L:G ratio of 50:50, 75:25, 85:15, etc., poly(lactic acid), polycaprolactone and fatty acid such as stearic acid and palmitic acid, and a mixture thereof, wherein the rate limiting polymer is present in a concentration of about 0.2% w/w to 50% w/w of total composition by weight.

The pharmaceutical composition according to the present invention, wherein, the naltrexone decanoate having a concentration in the range of 50 to 1500 mg/ml, is useful for treating opioid dependence and alcohol addiction in a patient in need thereof.

The pharmaceutical composition according to the invention can be administered as single administrable volume ranging from 0.5 to 5 ml by subcutaneous injection or intramuscular injection.

### DESCRIPTION OF DRAWING

FIG. 1: Blood plasma concentration of Naltrexone in rats after administration of different formulations of Naltrexone decanoate compositions (Formulation A, Formulation B and Formulation F).

FIG. 2: The chromatogram of naltrexone decanoate for the analysis of impurity using HPLC method.

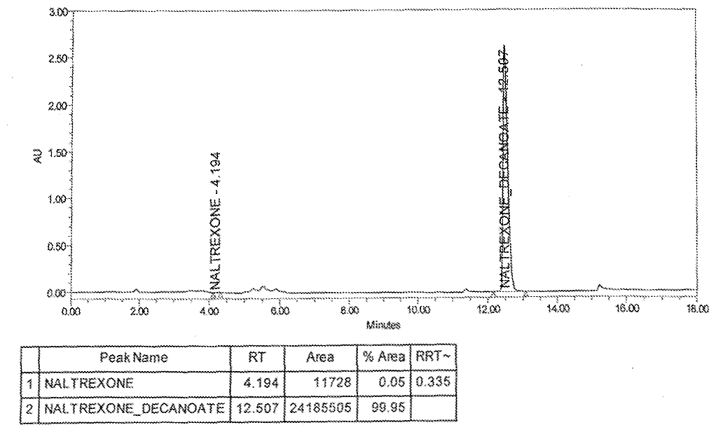

### DESCRIPTION OF THE INVENTION

Accordingly, the present invention describes an improved process for the synthesis of naltrexone decanoate with high purity and its sustained release pharmaceutical composition with the release of naltrexone ranging from 7 days to 90 days. The present invention further describes synthesis of Naltrexone decanoate and its pharmaceutically acceptable acid addition salts which includes organic and inorganic salts, more preferably organic salts. The present invention also describes the process for reconverting organic acid addition salt of naltrexone decanoate to naltrexone decanoate with substantial purity and yield by treating with a suitable base in a non-polar solvent.

The acids commonly employed to form acid addition salts may include but are not limited to, inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, decanoic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, phenylacetate, phenylpropionate, phenyibutyrate, citrate, lactate, gamma-propanesulfonate, naphthalene-1-sulfonate, naphthalene-2-sulfonate, mandelate, hydroxybutyrate, glycolate, tartrate, methanesulfonate and the like.

As used herein, the term “pharmaceutically acceptable salts” refer to derivatives wherein the parent compound, naltrexone decanoate is modified by making acid or base salts thereof. Suitable acceptable salts include, but are not limited to, mineral or organic acid salts of basic residues such as amines; alkali or organic salts of acidic residues such as carboxylic acids; and the like. The acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluene sulfonic, methane-sulfonic, ethane disulfonic, oxalic, isethionic, and the like. Specifically, the acceptable salts can include, for example, those salts that naturally occur in vivo in a mammal.

Preparation of Naltrexone Decanoate:

The process for preparation of naltrexone decanoate wherein the reaction is carried out in a monophasic medium using a single non-aqueous solvent, wherein the reaction involves esterifying the hydroxyl group of Naltrexone with decanoyl chloride which serves as an acylating agent in the presence of a base, wherein the base is an organic amine, preferably triethylamine or Hunig's base in the presence of a non-aqueous solvent which include, but are not limited to, ethyl acetate, cyclo-pentyl-methyl-ether, tetrahydrofuran, acetonitrile, acetone, dimethyl formamide and dichloromethane. Preferably the reaction is carried out by using the solvent, cyclopentyl methyl ether (CPME), cyclopentyl ethyl ether, cyclopentyl propyl ether.

The preparation of Naltrexone to Naltrexone decanoate, is shown in scheme 1.

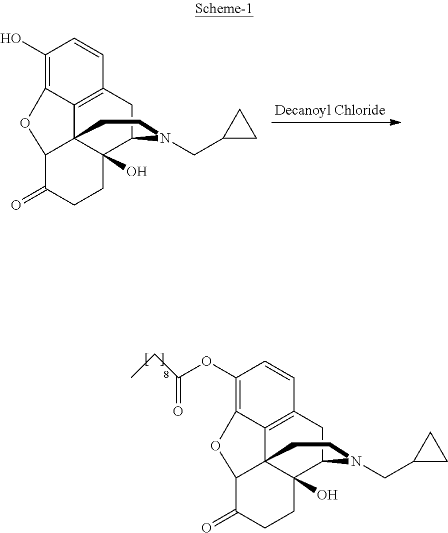

After completion of reaction, water was added to reaction mixture, then aqueous layer and organic layer were separated wherein the aqueous layer was separated with dichloromethane and the organic layer was washed with water and brine solution, dried over anhydrous magnesium sulphate or anhydrous sodium sulphate, and concentrated typically under reduced pressure, to provide naltrexone decanoate in the form of a viscous oil.

The naltrexone decanoate can be further purified by using any conventional methods well known to those skilled in the art including, but not limited to, column chromatography, high pressure liquid chromatography (HPLC), gas chromatography (GC), recrystallization, and/or distillation.

After the purification step, naltrexone decanoate is obtained with the levels of an impurity (0.5-1.5%), bis-decanoyl naltrexone. This impurity can be eliminated by preparing acid addition salts of naltrexone decanoate obtained by the above process and further reconverting into naltrexone decanoate from its organic acid addition salts by neutralizing the acid addition salts using a base in the presence of suitable solvent for obtaining substantially pure naltrexone decanoate with an HPLC purity of more than 99.6 to 99.9% with impurity levels between (0.05-0.3%).

Preparation of Organic Acid Addition Salts of Naltrexone Decanoate:

The organic acid addition salt of naltrexone decanoate is prepared by reacting naltrexone decanoate with an organic acid, in polar protic solvent, preferably n-propanol and non-polar solvent, preferably methyl-tertiary-butyl ether at a temperature in the range of 5° C. to 70° C., preferably in the range of 30° C. to 35° C. for a period of 20 minutes to 24 hours.

The synthesis of various organic acid addition salts of naltrexone decanoate, is shown in below scheme 2.

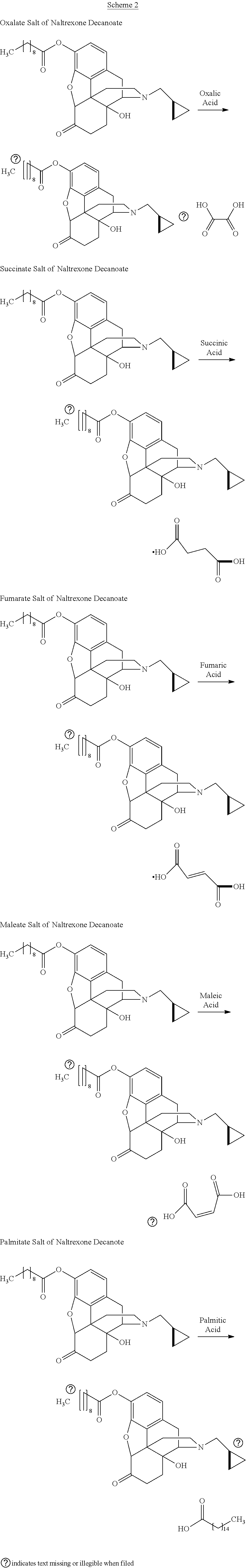

Decanoate Salt of Naltrexone Decanoate

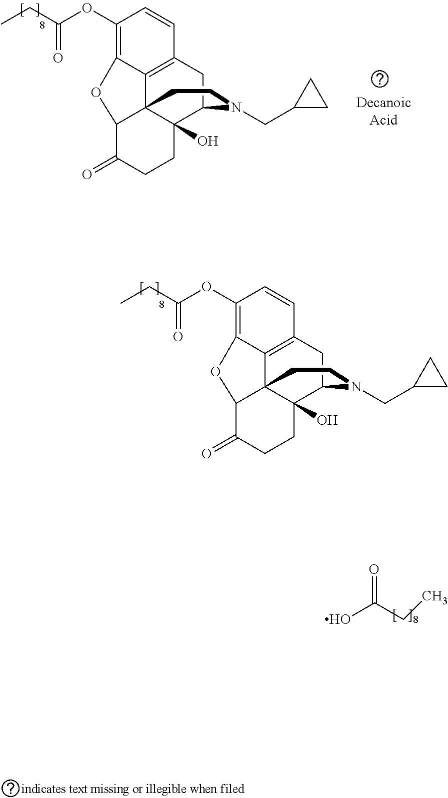

Further, the organic acid addition salt of naltrexone decanoate may be isolated by the steps of filtration, centrifugation, washing and drying.

Preparation of Highly Pure Naltrexone Decanoate from the Organic Acid Addition Salts of Naltrexone Decanoate:

The organic acid addition salt of naltrexone decanoate is converted into pure naltrexone decanoate by treating the organic acid addition salt of naltrexone decanoate with an inorganic base, in a solvent, at a temperature in the range of 10° C. to 35° C., preferably in the range of 30° C. to 35° C. for a period of 15 minutes to 1 hour.

Then organic layer was separated and washed with water. The resulting organic layer was dried over sodium sulphate and concentrated under reduced pressure to obtain pure naltrexone decanoate in the form of a viscous oil. The naltrexone decanoate is obtained in the yield of more than 93% and with an HPLC purity of about 99.9%.

The pure naltrexone decanoate obtained in the form of viscous oil with a purity of 99.9% is collected by filtration or centrifugation. The pure naltrexone decanoate compound is then optionally subjected to washing, re-crystallizing and drying using conventional drying techniques like vacuum oven drying.

Composition of Naltrexone Decanoate Sustained Release Injection:

Naltrexone decanoate obtained in the form of a viscous oil by the process can be formulated into a sustained release pharmaceutical composition by adding a pharmaceutically acceptable excipient, wherein the pharmaceutically acceptable excipient selected from vegetable oil such as cottonseed oil or sesame oil, an organic solvent selected from the group consisting of ethyl alcohol, benzyl alcohol, benzyl benzoate, N-methyl pyrrolidone and a mixture thereof and a release retarding agent selected from the group consisting of poly ortho esters such as poly(lactic-co-glycolic acid) of L:G ratio of 50:50, 75:25, 85:15, etc., poly(lactic acid) or polycaprolactone or fatty acid such as stearic acid and palmitic acid and a mixture thereof. This composition can be used for the treatment of opioid dependence, alcohol dependence, for a period of 7 days to 90 days in a patient in need thereof, wherein the composition provides sustained release of Naltrexone and can be administered by subcutaneous injection or intramuscular injection.

### EXAMPLES

### Example 1

Synthesis of Naltrexone Decanoate in Dichloromethane:-

To a stirred solution of Naltrexone HCl 2.5 g (7.3 mmol) in dichloromethane at 0-5° C. triethyl amine 0.91 g (9.15 mmol) was added. To above reaction mixture decanoyl chloride1.83 mL (8.7 mmol) was added at 0-5° C. The reaction mixture was stirred at 0-5° C. for 30 min. After completion of reaction, water was added to reaction mixture. After the reaction, the layers were separated and the aqueous layer was extracted with dichloromethane. The combined organic layer was washed with water, brine and dried on sodium sulphate. Further, evaporation of the solvent provided with an oil of naltrexone decanoate.

Yield: 3-3.21 gm (92-98%)

Purity: 98-99.9%

bis-decanoyl naltrexone impurity: 0.15-0.3

### Example 2

Synthesis of Naltrexone Decanoate in Cyclo-Pentyl-Methyl-Ether Solvent:-

To a stirred solution of Naltrexone HCl 25 g (66 mmol) in cyclo-pentyl-methyl-ether at 0° C. to 5° C. triethyl amine 16.7 g (165 mmol) was added. To above reaction mixture decanoyl chloride13.5 mL (65.3 mmol) was added at 0-5° C. The reaction mixture was at 0-5° C. for 30 min. After completion of reaction, water was added to reaction mixture. The aqueous and organic layers were separated; aqueous layer was extracted with cyclo-pentyl-methyl-ether. The combined organic layer was washed with water, brine and dried on sodium sulphate. Further, evaporation of the solvent provided with an oil of naltrexone decanoate.

Yield: (92-98%)

Purity: 98-99.8%

bis-decanoyl naltrexone Impurity: 0.15-0.3%

### Example 3

Synthesis of Naltrexone Decanoate Oxalate Salt:-

To a stirred solution of Naltrexone decanoate 10.0 g (20.2 mmol) in methyl-tert-butyl-ether (80 mL), n-propanol 20 mL was added at room temperature. Prepared a solution of oxalic acid 1.81 g in methyl-tert-butyl ether (60 mL) and n-propanol(10 mL). The above solution was added to a mixture of naltrexone decanoate at room temperature. Stirred the reaction mixture at room temperature for 16 hr. Filtered the precipitates obtained and gave washings with methyl-tert-butyl ether (50 mL). Dried the solid under vacuum at 65° C. till constant weight is obtained. The yield obtained is 6.4 g (54%).

### Example 4

Synthesis of Naltrexone Decanoate Maleate Salt:-

To a stirred solution of Naltrexone decanoate 2.0 g (4.0 mmol) in methyl-tert-butyl ether(16 mL) was added n-propanol (2 mL) at room temperature. Prepared a solution of maleic acid 0.46 g (4.0 mmol) in methyl-tert-butyl ether(12 mL) and n-propanol(1 mL). The above solution was added to a mixture of naltrexone decanoate at room temperature. Heated the above reaction mixture at 50-55° C. for 30 min. Cooled the reaction mixture to 0-5° C. gradually, white precipitate obtained. Filtered the precipitates obtained and gave washings with methyl-tert-butyl ether(10 mL). Dried the solid under vacuum at 55° C. till constant weight is obtained. The yield obtained is 1.28 gm to 1.54 g (54-65%)

### Example 5

Preparation of Pure Naltrexone Decanoate from Acid Addition Salt of Naltrexone Decanoate:-

A solution of acid addition salt (Oxalate salt) of Naltrexone decanoate (25 gm) in dichloromethane (250 ml) was added (7-8%) aqueous solution of sodium bicarbonate (170 ml) up to pH 8.0 at 10-15° C. and then resulting reaction mixture was stirred for 30 minutes at 25-30° C. Then organic layer was separated and washed with water. The resulting organic layer was dried over sodium sulphate and concentrated under reduced pressure to get pure Naltrexone decanoate as yellow viscous oil. The yield obtained is 3.4 g (93%) and with the purity of about 99.9%.

MS:- m/z 496.7(M+)

Bis-Decanoyl Naltrexone Impurity: NIL.

In similar manner, the pure Naltrexone decanoate was prepared from other maleate salt of Naltrexone decanoate, with bis-decanoyl naltrexone Impurity in undetectable limits.

### Example 6

Composition of Naltrexone Decanoate Sustained Release Injection:-

100 ml sesame oil was taken and 41.84 g Naltrexone decanoate (276 mg/ml) slowly

added with continuous stirring at 800 rpm for 35 min using magnetic stirrer. The drug was miscible with sesame oil and then 1.81 g benzyl alcohol (12 mg/ml) was added to drug solution with continuously stirring at 800 rpm for 1 hr. using magnetic stirring, then the volume was made up to 150 ml using sesame oil to obtain a clear solution. This solution was filtered using 0.45 um PTFE filter by vacuum

filtration, then the formulation was stored at 2-8° C. This formulation (FormulationA) was administered by SC injection in Wistar rats with the dose equivalent to 100 mg/kg Naltrexone. The blood plasma concentration of active metabolite,Naltrexone observed was above 2 ng/mL till 14 days.

### Example 7

Composition of Naltrexone Decanoate Sustained Release Injection:-

A formulation containing 276 mg/mL Naltrexone decanoate, 100 mg/mL PLGA in N-Methylpyrrolidone (0.44 ml) & Benzyl benzoate (0.46 mL) (Formulation B) wastested in-vivo in Wistar rats after subcutaneous injection with the dose equivalent

to Naltrexone 100 mg/kg. The formulation of naltrexone decanoate resulted into naltrexone with the blood plasma concentration of above 1 ng/mL till 28 days. A quantifiable amount of Naltrexone was also observed till 49 days in these rats.

### Example 8

Composition of Naltrexone Decanoate Sustained Release Injection:-

A composition containing 736 mg/mL Naltrexone decanoate in N-Methylpyrrolidone (quantity sufficient to 1 ml) (Formulation F) resulted into about 6.5 ng/mL on 15th day post-administration, in rat blood plasma after administration of dose equivalent to Naltrexone 100 mg/kg by intramuscular route.

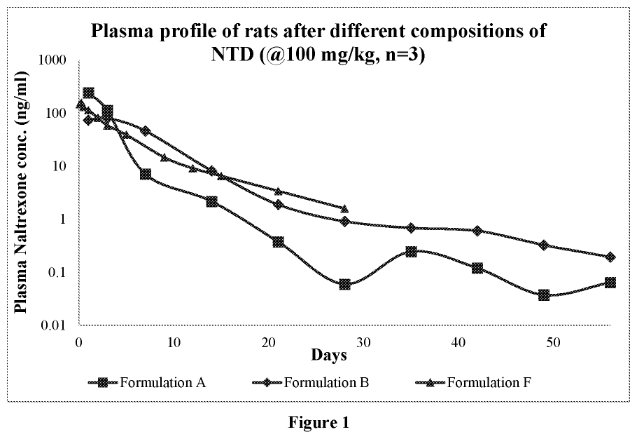

FIG. 1 shows the naltrexone plasma concentrations in rats after a single subcutaneous or Intramuscular injection of naltrexone decanoate composition equivalent to Naltrexone 100 mg/kg dose. It is apparent from the figure that the in vivo drug release of active moiety, Naltrexone after the injection of Naltrexone decanoate occurred in a controlled manner. After the injection of naltrexone decanoate prepared with sesame oil and benzyl alcohol (Formulation A), the bloodplasma concentration of naltrexone observed was above 2 ng/mL till 14 days and after the injection of Formulation B of naltrexone decanoate, prepared with PLGA in N-Methylpyrrolidone, the blood plasma concentration of active moiety, Naltrexone observed was above 1 ng/mL till 28 days and a quantifiable amount of Naltrexone was also observed till 49 days in these rats. After the injection of Formulation F of naltrexone decanoate, prepared in N-Methylpyrrolidone, the blood plasma concentration of active moiety, Naltrexone was observed about 6.5 ng/mL on 15th day post-administration.

1-20. (canceled)

21. A process for the preparation of an ester of naltrexone, comprising the steps of:
a) reacting naltrexone or a salt thereof with an acylating agent in the presence of an organic base and an organic solvent to obtain an ester of naltrexone,
wherein the acylating agent is a halide of a saturated or unsaturated carboxylic acid having 2-18 carbon atoms,
wherein the ester of naltrexone comprises a saturated or unsaturated acyl moiety having 2-18 carbon atoms;
b) converting the ester of naltrexone into an acid addition salt by treating the ester of naltrexone with an acid in an organic solvent; and
c) neutralizing the acid addition salt by treating the acid addition salt with a base to isolate a purified ester of naltrexone.

22. The process as claimed in claim 21, wherein the step of reacting naltrexone or the salt thereof with an acylating agent is carried out in the presence of an organic base selected from the group consisting of triethylamine, diisopropylethylamine, tripropyl amine, trimethyl amine, N-methyl pyrrolidine, pyridine, N-methyl morpholine, N,N-dimethyl aminopyridine, and mixtures thereof.

23. The process as claimed in claim 21, wherein the step of reacting naltrexone or the salt thereof with an acylating agent is carried out in the presence of an organic solvent selected from the group consisting of
a non-polar organic solvent selected from the group consisting of dichloromethane, diethyl ether, methyl tertiary-butyl ether, cyclopentyl methyl ether, 1,4-dioxane, toluene, pentane, cyclopentane, hexane, cyclohexane, heptane, and mixtures thereof;
a polar organic solvent selected from the group consisting of methanol, ethanol, n-butanol, isopropanol, n-propanol, ethyl acetate, tetrahydrofuran, dichloromethane, acetonitrile, acetone, dimethyl formamide, and mixtures thereof; and
mixtures thereof.

24. The process as claimed in claim 21, wherein the step of reacting naltrexone or the salt thereof with an acylating agent is carried out in the presence of the non-polar organic solvent, wherein the non-polar organic solvent is cyclopentyl methyl ether.

25. The process as claimed in claim 21, wherein:
the step of reacting naltrexone or the salt thereof with an acylating agent is carried out at a temperature between 0° C. and 1100° C.; and
the step of reacting naltrexone or the salt thereof with an acylating agent is carried out for a period between 20 minutes and 48 hours.

26. The process as claimed in claim 21, wherein the acylating agent is selected from the group consisting of decanoyl chloride, palmitoyl chloride, stearoyl chloride, myristoyl chloride sebacoyl chloride, acetyl chloride, propionyl chloride, butyryl chloride, valeryl chloride, hexanoyl chloride, heptanoyl chloride, octanoyl chloride, nonanoyl chloride, undecanoyl chloride, dodecanoyl chloride, tridecanoyl chloride, pentadecenoyl chloride, heptadecanoyl chloride, isobutyroyl chloride, and mixtures thereof.

27. The process as claimed in claim 21, wherein converting the ester of naltrexone into the acid addition salt comprises treating the ester of naltrexone with an acid selected from the group consisting of acetic acid, propionic acid, glycolic acid, pyruvic acid, oxalic acid, decanoic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluene sulfonic acid, salicylic acid, and mixtures thereof.

28. The process as claimed in claim 21, wherein the step of neutralizing the acid addition salt comprises treating the acid addition salt with an inorganic base in a non-polar organic solvent at a temperature in the range of 10° C. to 35° C.;
wherein the inorganic base is selected from the group consisting of sodium bicarbonate, sodium carbonate, potassium bicarbonate, potassium carbonate, sodium hydroxide and potassium hydroxide, lithium bicarbonate, lithium carbonate, lithium hydroxide, calcium hydroxide, barium hydroxide, and mixtures thereof.

29. The process as claimed in claim 21, wherein the ester of naltrexone is selected from the group consisting of naltrexone acetate, naltrexone butyrate, naltrexone velarate, naltrexone hexanoate, naltrexone heptanoate, naltrexone octanoate, naltrexone nonaoate, naltrexone undecanoate, naltrexone dodecanoate, naltrexone tridecanoate, naltrexone myristate, naltrexone pentadecanoate, naltrexone palmitate, naltrexone heptadecanoate, naltrexone isobutyrate, naltrexone pivaloate, naltrexone propionate, naltrexone stearate, naltrexone decanoate, and naltrexone sebacate, and mixtures thereof.

30. The process as claimed in claim 21, wherein:
the ester of naltrexone is naltrexone decanoate; and
the acid addition salt is selected from the group consisting of naltrexone decanoate hydrochloride, naltrexone decanoate oxalate, naltrexone decanoate succinate, naltrexone decanoate tartrate, naltrexone decanoate maleate and naltrexone decanoate decanoate, and mixtures thereof.

31. A process for the preparation of naltrexone decanoate, comprising the steps of:
a) reacting naltrexone or a salt thereof with a halide of decanoic acid to obtain naltrexone decanoate;
b) converting the naltrexone decanoate into an acid addition salt by treating the ester of naltrexone with an acid in an organic solvent; and
c) neutralizing the acid addition salt by treating the acid addition salt with a base to isolate purified naltrexone decanoate in a yield of >90%;
wherein the naltrexone decanoate has a purity of >98% and is devoid of bis-decanoyl naltrexone.

32. The process as claimed in claim 31, wherein the naltrexone decanoate has a purity of >98% after storage for a period of up to 60 months at a storage temperature of less than 25° C.

33. A sustained release pharmaceutical composition, comprising an ester of naltrexone,
wherein the ester of naltrexone is produced by the method of claim 1,
wherein a single dose of the sustained release pharmaceutical composition delivers naltrexone to a patient in need thereof for over a period ranging from 7 days to 90 days.

34. The sustained release pharmaceutical composition as claimed in claim 33, wherein the ester of naltrexone is selected from the group consisting of naltrexone acetate, naltrexone butyrate, naltrexone velarate, naltrexone hexanoate, naltrexone heptanoate, naltrexone octanoate, naltrexone nonaoate, naltrexone undecanoate, naltrexone dodecanoate, naltrexone tridecanoate, naltrexone myristate, naltrexone pentadecanoate, naltrexone palmitate, naltrexone heptadecanoate, naltrexone isobutyrate, naltrexone pivaloate, naltrexone propionate, naltrexone stearate, naltrexone decanoate and naltrexone sebacate, and mixtures thereof, having a concentration in the range of 50 to 1500 mg/ml.

35. The sustained release pharmaceutical composition as claimed in claim 33, further comprising at least one pharmaceutical excipient selected from the group consisting of:
a) a vehicle in a concentration of about 30.0% w/w to 90.0% w/w of the total composition;
b) a stabilizer in a concentration of about 0.01% w/w to 30.0% w/w of the total composition; and
c) a rate limiting polymer in a concentration of about 0.2% w/w to 50% w/w of the total composition.

36. The sustained release pharmaceutical composition as claimed in claim 35, further comprising the vehicle, wherein the vehicle is selected from the group consisting of vegetable oils, water miscible organic solvents, water immiscible organic solvents, and mixtures thereof.

37. The sustained release pharmaceutical composition as claimed in claim 35, further comprising the stabilizer, wherein the stabilizer is selected from the group consisting of benzyl alcohol, butylated hydroxyl anisole (BHA), butylated hydroxyl toluene (BHT), alpha-tocopherol, and mixtures thereof.

38. The sustained release pharmaceutical composition as claimed in claim 35, further comprising the rate limiting polymer, wherein the rate limiting polymer is selected from the group consisting of:
poly(lactic acid-co-glycolic acid), wherein the Lactide:Glycolide ratio is between 50:50 and 85:15;
poly(lactic acid);
polycaprolactone;
a fatty acid; and
mixtures thereof.

39. A method for treating a patient addicted to an opioid and/or alcohol, comprising administering an effective amount of the sustained release pharmaceutical composition as claimed in claim 33 to the patient.

40. The method of claim 39, wherein the sustained release pharmaceutical composition is administered by subcutaneous or intramuscular injection in a volume ranging from 0.5 to 5 ml.
